(19) United States
(12) Patent Application Publication
(10) Pub. No.: US 2010/0210962 A1
(43) Pub. Date: Aug. 19, 2010

(54) Respiratory signal detection and time domain signal processing method and system

(76) Inventors: Jingping Xu, Vancouver, WA (US); Deepak Ayyagari, Vancouver, WA (US); Yongji Fu, Aloha, OR (US)

Correspondence Address: Scot Reader, Suite 228, 1320 Pearl Street, Boulder, CO, 80302, US

(21) Appl. No.: 12/378,476

(22) Filed: Feb. 13, 2009

(51) Int. Cl. A61B 5/08

(52) U.S. Cl. 600/529

(57) ABSTRACT

A respiratory signal detection and time domain signal processing method and system classifies respiratory phases and determines respiratory time data useful in respiratory health determinations. The method and system analyze respiratory signals collected at multiple detection points at least one of which ensures that respiratory phases can be properly classified. Moreover, the method and system employ a time domain signal processing approach that facilitates determination of respiratory time data while realizing savings in computing power relative to frequency domain processing approaches.

### BACKGROUND OF INVENTION

The present invention relates to respiratory health and, more particularly, to a respiratory signal detection and time domain signal processing method and system that classifies respiratory phases and determines fractional respiratory time data useful in respiratory health determinations.

Respiration in humans is typically characterized by two phases: inspiration, or the intake of air into the lungs, and expiration, or the expelling of air from the lungs. Respiratory time data that characterize these respiratory phases is very important in individual respiratory health determinations and the study of pulmonary diseases. For example, a low fractional inspiratory time (i.e. inspiratory time divided by respiratory period) may reflect a prolonged expiratory phase that is indicative of obstruction of the airways. A high fractional inspiratory time may inform as to the present status of a monitored subject, for example, that the subject is snoring or speaking.

Generally speaking, there are three methods for obtaining respiratory time data. One is the respiratory airflow method. In this method, the subject breathes into an apparatus that measures the airflow through the subject's mouth. This method can provide reliable respiratory time data. However, this method is inconvenient and episodic as the person must place the apparatus next to his or her mouth. This method is not suitable for consumer-friendly and continuous respiratory monitoring.

Another method is respiratory inductance plethysmography (RIP). In this method, the subject wears a first inductance band around his or her ribcage and a second inductance band around his or her abdomen. As the subject breathes, the volumes of the ribcage and abdominal compartments change which alters the inductance of coils. Respiratory time data are determined based on the changes in inductance. This method has a disadvantage in that it requires quantitative calibration. Additionally, it is often difficult to achieve stable positioning of the inductance bands on subjects with poor postural control or physical deformities.

Yet another method is the lung sound method, sometimes called auscultation. The lung sound method has become increasingly popular due in part to the low cost and ready availability of lung sound detection systems. In this method, a respiratory sound transducer generates an acoustic respiratory signal from which respiratory time data are determined. One problem with known implementations of the lung sound method is over-reliance on tracheal sound transducers. Tracheal sound transducers, typically placed over the suprasternal notch or at the lateral neck near the pharynx, are often chosen for respiratory signal detection because such respiratory signals have a high signal-to-noise ratio and a high sensitivity to variation in flow that enable accurate demarcation of respiratory phase starting points. However, the difference in amplitude between inspiratory and expiratory tracheal respiratory signals varies greatly among subjects. For many people, inspiratory sounds are louder while for others there is not much difference and for still others expiratory sounds are louder. Therefore, distinguishing between the inspiratory and expiratory phases can be difficult using tracheal respiratory signals alone. Another shortcoming of known implementations of the lung sound method is reliance on frequency domain signal processing that uses spectral analysis to distinguish between inspiratory and expiratory phases and requires substantial computing power.

### SUMMARY OF THE INVENTION

The present invention, in a basic feature, provides a respiratory signal detection and time domain signal processing method and system that classifies respiratory phases and determines respiratory time data useful in respiratory health determinations. The present method and system analyze respiratory signals collected at multiple detection points at least one of which ensures that respiratory phases can be properly classified. For example, since inspiration sound detected at the chest of most human subjects exceeds expiration sound by about 6-10 dB across a large frequency range, a first respiratory sound transducer may be placed at the chest of a subject being monitored to ensure that inspiratory and expiratory phases can be accurately identified. The first respiratory sound transducer may compliment a second respiratory sound transducer placed at the trachea of the subject that ensures accurate demarcation of respiratory phase starting points. Moreover, the present method and system employ a time domain signal processing approach that facilitates determination of respiratory time data while realizing savings in computing power relative to frequency domain signal processing approaches.

In one aspect of the invention, a time domain signal processing method comprises the steps of determining starting points for a plurality of respiratory phases using a tracheal respiratory signal and classifying the respiratory phases into inspiratory and expiratory phases using the starting points and a chest respiratory signal.

In some embodiments, the method further comprises the step of determining respiratory time data using the starting points and the classified respiratory phases.

In some embodiments, the respiratory time data comprise one or more of average inspiratory time or average fractional inspiratory time.

In some embodiments, the method further comprises the step of applying a band-pass filter to the tracheal respiratory signal.

In some embodiments, the method further comprises the step of applying a smooth finite impulse response (FIR) filter to the tracheal respiratory signal.

In some embodiments, the method further comprises the step of down-sampling the tracheal respiratory signal.

In some embodiments, the method further comprises the step of applying an autocorrelation function to the tracheal respiratory signal.

In some embodiments, the method further comprises the step of applying a band-pass filter to the chest respiratory signal.

In some embodiments, the method further comprises the step of applying a smooth FIR filter to the chest respiratory signal.

In some embodiments, the method further comprises the steps of collecting and amplifying the tracheal respiratory signal and the chest respiratory signal.

In another aspect of the invention, a time domain signal processing method comprises the steps of determining starting points for a plurality of respiratory phases using a respiratory signal from a first body position, classifying the respiratory phases into inspiratory and expiratory phases using the starting points and a respiratory signal from a second body position and determining respiratory time data using the starting points and the classified respiratory phases.

In some embodiments, the first body position is the trachea and the second body position is the chest.

In some embodiments, the respiratory time data comprise one or more of average inspiratory time or average fractional inspiratory time.

In yet another aspect of the invention, a respiratory signal detection and signal processing system comprises first and second respiratory sound transducers adapted to collect first and second collected respiratory signals from first and second body positions, respectively; and a time domain signal processor adapted to receive first and second received respiratory signals based on the first and second collected respiratory signals, respectively, wherein the time domain signal processor determines starting points for a plurality of respiratory phases using the first received respiratory signal, classifies the respiratory phases into inspiratory and expiratory phases using the starting points and the second received respiratory signal and determines respiratory time data using the starting points and the classified respiratory phases.

In some embodiments, the first collected respiratory signal comprises a tracheal respiratory signal and the second collected respiratory signal comprises a chest respiratory signal.

In some embodiments, the respiratory time data comprise one or more of average inspiratory time or average fractional inspiratory time.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a respiratory signal detection system in which the invention is operative in some embodiments.

FIG. 2 shows a contemporaneous tracheal respiratory signal and chest respiratory signal.

FIG. 3 shows a tracheal respiratory signal after application of a band-pass filter and a tracheal respiratory signal envelope after application of a smooth FIR filter.

FIG. 4 shows an autocorrelation function for a tracheal respiratory signal envelope.

FIG. 5 shows various tracheal respiratory signal envelopes after application of various smooth FIR filters.

FIG. 6 shows a tracheal respiratory signal envelope after application of a high-order FIR filter with peaks of respiratory phases marked.

FIG. 7 shows various tracheal respiratory signal envelopes after application of various FIR filters with starting points of respiratory phases marked.

FIG. 8 shows a tracheal respiratory signal with starting points of respiratory phases marked.

FIG. 9 shows a chest respiratory signal with starting points of respiratory phases marked.

FIG. 10 shows a chest respiratory signal after application of a band-pass filter with starting points of respiratory phases marked.

FIG. 11 shows an envelope for a chest respiratory signal after application of a smooth FIR filter.

FIG. 12 shows median values of envelope sections corresponding to respiratory phases for a chest respiratory signal.

FIG. 13 shows a time domain signal processing method that classifies respiratory phases and determines respiratory time data in some embodiments of the invention.

### DETAILED DESCRIPTION OF A PREFERRED EMBODIMENT

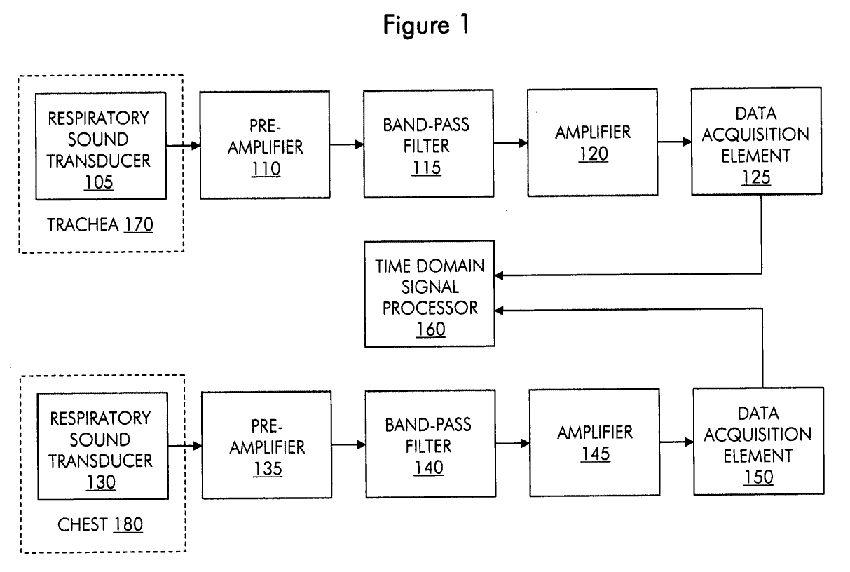

FIG. 1 shows a respiratory signal detection system in which the invention is operative in some embodiments. The system includes a first respiratory sound transducer 105 positioned at the trachea 170 of a human subject being monitored. Transducer 105 is communicatively coupled in series with a pre-amplifier 110, bond-pass filter 115, amplifier 120 and data acquisition element 125. The system also includes a second respiratory sound transducer 130 positioned at the chest 180 of the subject. Transducer 130 is communicatively coupled in series with a pre-amplifier 135, band-pass filter 140, amplifier 145 and a data acquisition element 150. Data acquisition elements 125, 150 transmit respiratory signals collected from transducers 105, 130 as modified by amplifiers 110, 120, 135, 145 and filters 115, 140 to a time domain signal processor 160. Time domain signal processor 160 may be collocated with one or more of the other elements shown in FIG. 1, may be a stand-alone element, or may be located remotely from the other elements shown in FIG. 1. Where time domain signal processor 160 is located remotely from the other elements, processor 160 may be coupled with such elements via a wireless link. In some embodiments, one of the respiratory sound transducers may be positioned at a different body location, such as the patient's back.

Transducers 105, 130 detect acoustic respiratory signals at respective detection points, namely trachea 170 and chest 180. Transducers 105, 130 provide high sensitivity, a high signal-to-noise ratio and a generally flat frequency response in the bond for lung sounds. Transducers 105, 130 in some embodiments are omni-directional piezo ceramic microphones. Microphones marketed by Knowles Acoustics as part BL-21785 may be used by way of example. Transducers 105, 130 output detected respiratory signals to respective pre-amplifiers 110, 135 as analog voltages on the order of 10-200 mV.

Pre-amplifiers 110, 135 provide impedance match for the respiratory signals received from transducers 105, 130 and amplify the respiratory signals to a level appropriate for the filter stage that follows. Pre-amplifiers marketed by Presonus Audio Electronics as TubePre Single Channel Microphone Preamp with VU (Volume Unit) Meter may be used by way of example.

Band-pass filter 115, 140 is an analog filter that applies a high-pass cutoff frequency at 80 Hz and a low-pass cutoff frequency at 2 KHz to the respiratory signals received from pre-amplifiers 110, 135 reduce noise, for example, heart sounds, muscle and contact noise.

Final amplifiers 120, 145 amplify the respiratory signals received from filters 115, 140 to the range of ±1 V.

Data acquisition elements 125, 150 perform A/D conversion on the respiratory signals received from amplifiers 120, 145 and down-sample the respiratory signals by a factor of four from an original sampling frequency of 44.1 KHz to a new sampling frequency of 8820 Hz in order to reduce the sampled data length. Data acquisition elements 125, 150 transmit the tracheal respiratory signal and chest respiratory signal, respectively, to time domain signal processor 160 for analysis.

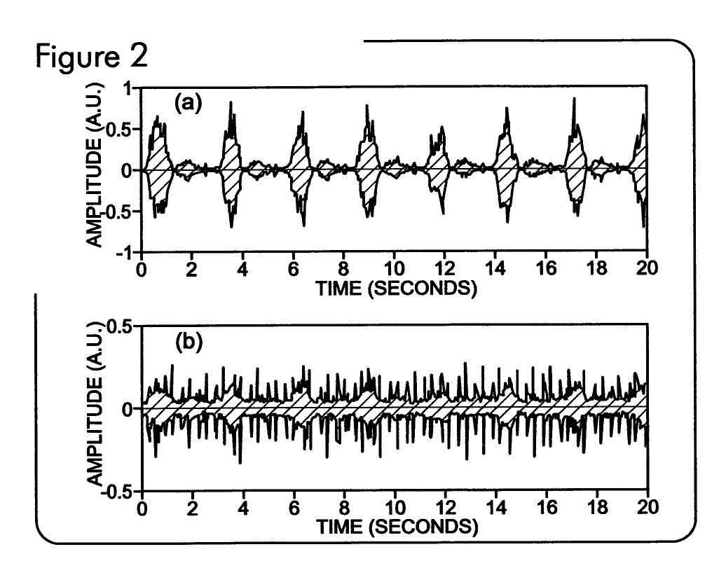

Time domain signal processor 160 is a microprocessor having software executable thereon for performing time domain signal processing on the tracheal respiratory signal received from data acquisition element 125 and the chest respiratory signal received from data acquisition element 150. The time domain signal processing classifies respiratory phases and determines respiratory time data. In some embodiments, processor 160 receives the tracheal respiratory signal and the chest respiratory signal and generates respiratory time data based thereon on a continuous basis to enable real-time monitoring of the respiratory health of a human subject. FIG. 2 shows contemporaneous tracheal respiratory signal and chest respiratory signal received by time domain signal processor 160, wherein the tracheal respiratory signal is shown under the heading “(a)” and the chest respiratory signal is shown under the heading “(b)”.

A time domain signal processing method will now be described by reference to the flow diagram in FIG. 13 taken in conjunction with the charts of FIGS. 2-12. First, a band-pass filter is applied to the tracheal respiratory signal received from data acquisition element 125 (1305). The band-pass filter has a cutoff frequency of 150 Hz at the low end and 850 Hz at the high end.

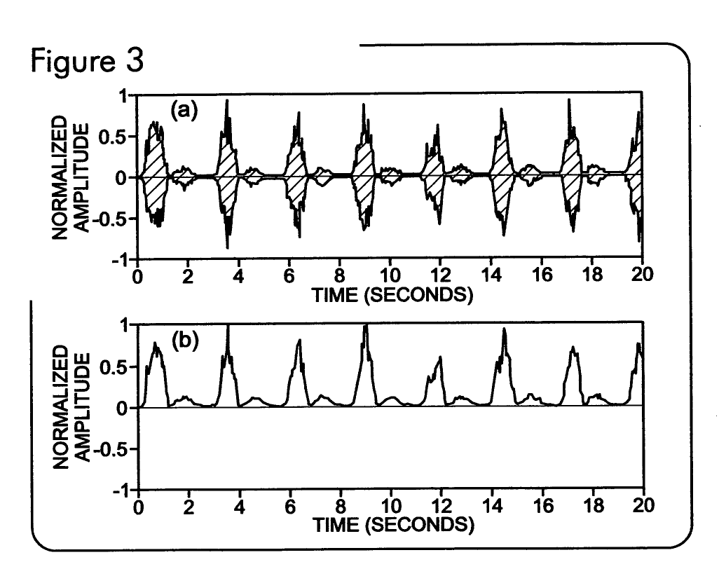

Next, a smooth FIR filter with order in the range of 800 to 1200 is applied to the tracheal respiratory signal to generate a smooth tracheal respiratory signal envelope (1310). The smooth FIR filter removes noise from the signal and improves signal quality. In some embodiments, the smooth FIR filter is a Hanning (Hann) window with order of 1000. FIG. 3 shows the tracheal respiratory signal after application of the band-pass filter in Step 1305 under heading “(a)” and the tracheal sound respiratory signal envelope after application of the smooth FIR filter in Step 1310 under heading “(b)”.

Next, the tracheal respiratory signal envelope is down-sampled to reduce the length of the envelope (1315), after which a low-order smooth FIR filter with order in the range of 250 to 350 is applied to further smooth the envelope (1320). In some embodiments, the low-order smooth FIR filter is a Hanning window of order 300.

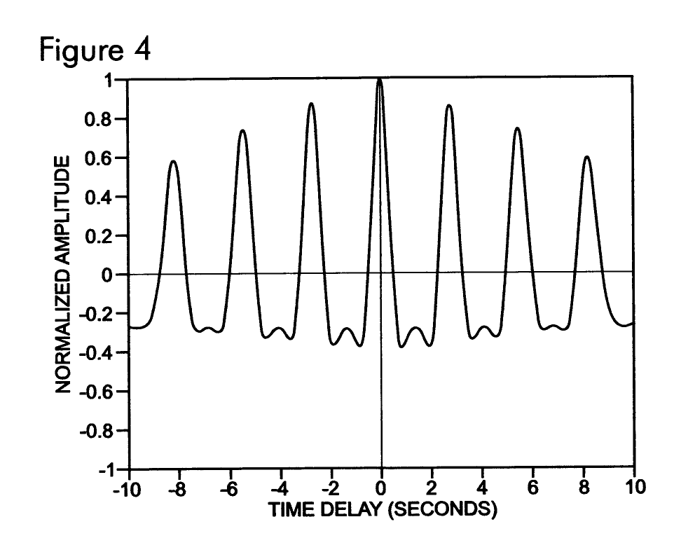

Next, an autocorrelation function is applied to the envelope (1325) to identify the fundamental periodicity of the time domain data. FIG. 4 shows the autocorrelation function for the tracheal respiratory signal envelope.

Next, average respiratory period is determined using the autocorrelated tracheal respiratory signal envelope (1330), wherein each respiratory period includes two contiguous respiratory phases (i.e. one inspiratory phase and one expiratory phase). In some embodiments, the average respiratory period is identified as the peak-to-peak time difference between the highest peak and the next peak of similar amplitude in the positive or negative direction within the tracheal respiratory signal envelope. Returning to FIG. 4, for example, the time difference between the highest peak and the next peak of similar amplitude in the positive direction is 2.74 seconds, which may be identified and applied as the average respiratory period.

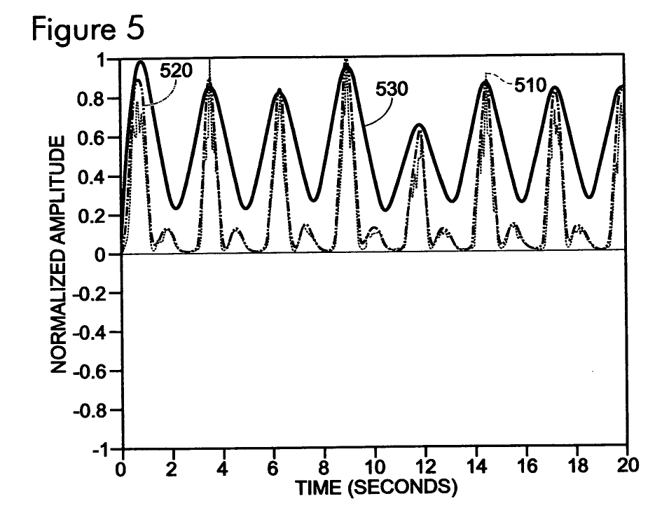

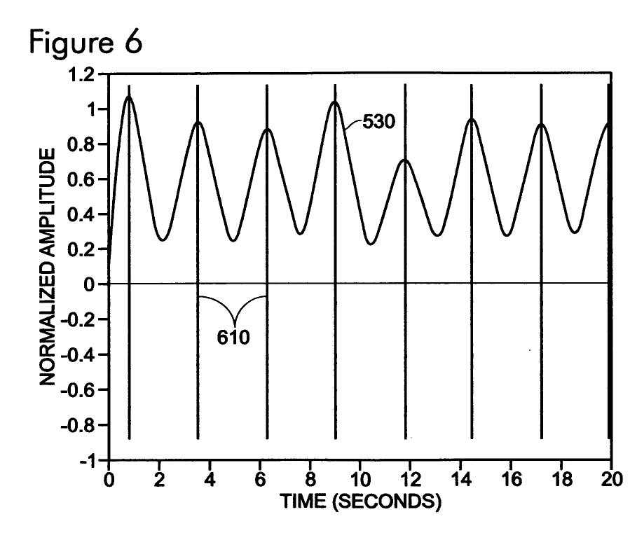

Next, a high-order smooth FIR filter is applied to further smooth the envelope (1335). After application, the envelope contains only one peak for each respiratory period. The order of the high-order smooth FIR filter is adaptive and is selected based on the average respiratory period determined in Step 1330. FIG. 5 shows the various tracheal respiratory signal envelopes after application of various smooth FIR filters. Before application of the low-order filter in Step 1320, envelope 510 is the least smooth. Smoothness is increased through application of the low-order filter in Step 1320 that produces a low-order smoothed envelope 520, and is further increased through application of the high-order filter in Step 1335 that produces a high-order smoothed envelope 530. FIG. 6 shows high-order smoothed envelope 530 with peaks of respiratory periods (e.g. 610) marked.

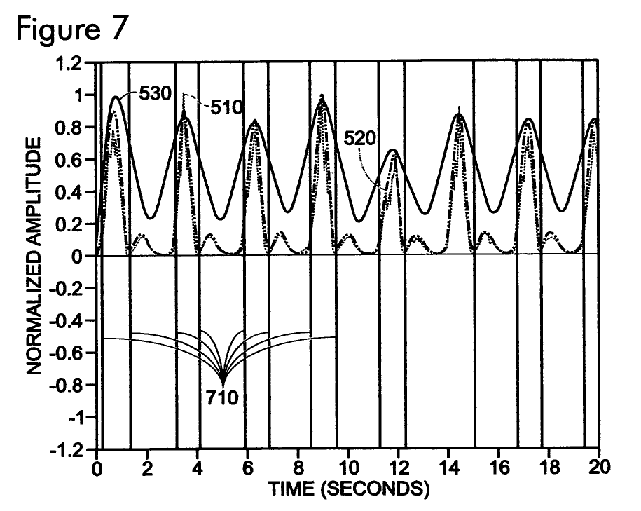

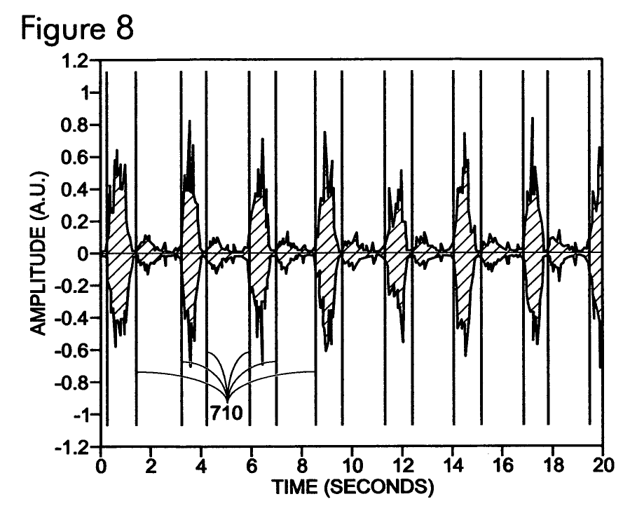

Next, the starting points of respiratory phases are determined using the low-order smoothed envelope 520 and high-order smoothed envelope 530 (1340). A starting point for each respiratory phase is identified as the time at which a rising amplitude on low-order smoothed envelope 520 has reached 10% of a corresponding peak amplitude on low-order smoothed envelope 520, wherein peak times of high-order smoothed envelope 530 are used to identify the peak amplitudes on low-order smoothed envelope 520. FIG. 7 shows various smoothed tracheal respiratory signal envelopes 510, 520, 530 with starting points of respiratory phases (e.g. 710) marked. For comparison, FIG. 8 shows the tracheal respiratory signal as originally received by time domain signal processor 160 with starting points of respiratory phases (e.g. 710) marked.

It bears noting that to this point respiratory phases have not yet been classified into inspiratory and expiratory phases. Such classification awaits analysis of the chest respiratory signal in Steps 1345 to 1355, which is now discussed in greater detail.

First, a band-pass filter is applied to the chest respiratory signal received from data acquisition element 150 (1345). The band-pass filter has a cutoff frequency of 100 Hz at the low end and 450 Hz at the high end.

Next, a smooth FIR filter with order in the range of 800 to 1200 is applied to the chest respiratory signal to generate a smooth chest respiratory signal envelope (1350). In some embodiments, the smooth FIR filter is a Hanning window with order of 1000.

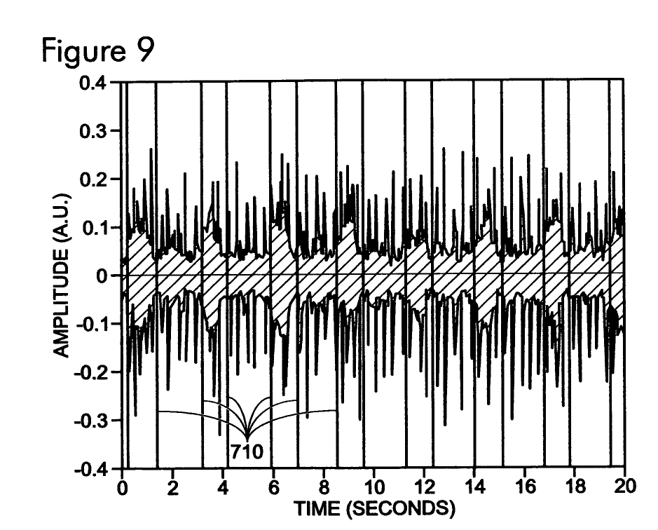

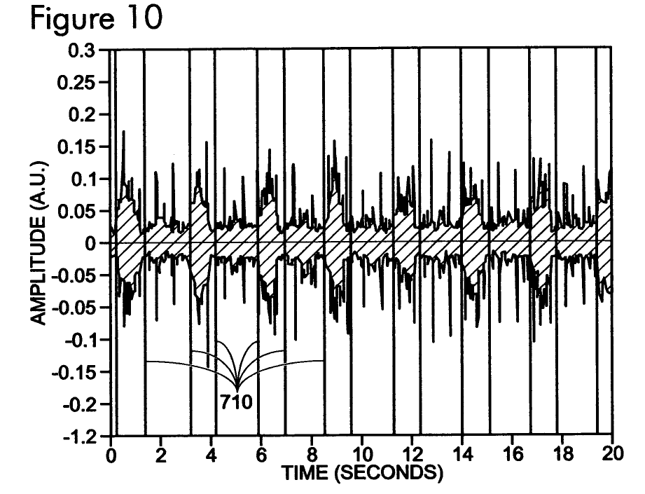

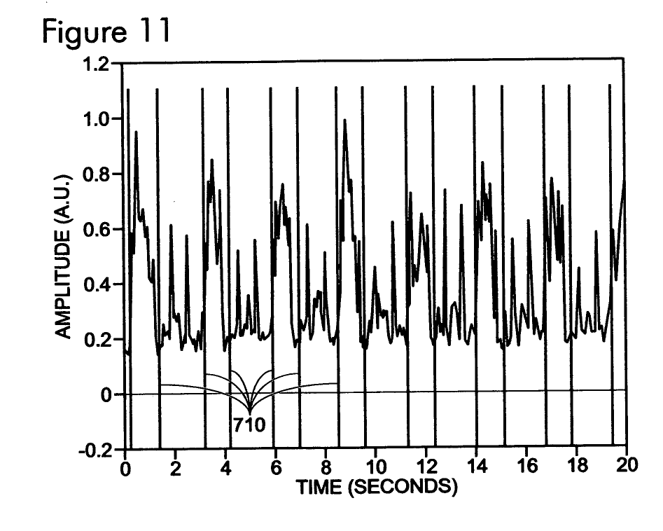

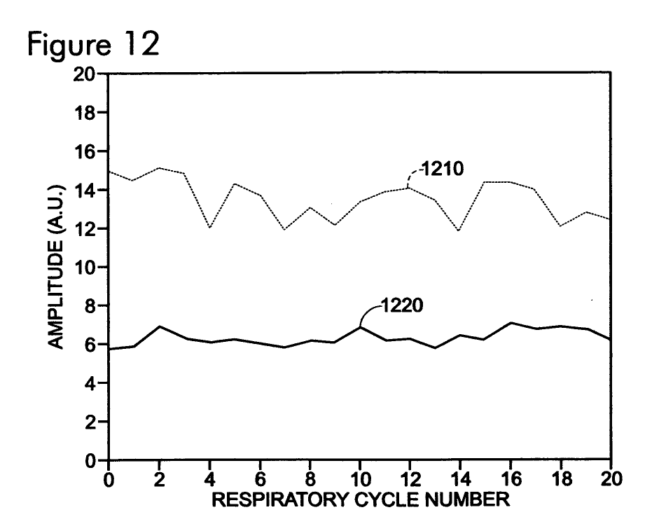

Next, using the starting points determined in Step 1340, respiratory phases are classified as either inspiratory or expiratory using the chest respiratory signal envelope (1355). Respiratory phases are first segmented into respiratory periods, with each period consisting of two contiguous phases. The earlier phases of each period are assigned to a first phase group and the later phases of each period are assigned to a second phase group. Median amplitudes are then calculated for the individual phases after which group averages are calculated for the first and second phase groups. The phases in the group that has the higher average are classified as inspiratory and the phases in the group that has the lower average are identified as expiratory. FIG. 9 shows the chest respiratory signal as originally received by time domain signal processor 160 with starting points of respiratory phases determined in Step 1340 (e.g. 710) marked. For comparison, FIG. 10 shows the chest respiratory signal after application of a band-pass filter in Step 1345 with starting points of respiratory phases determined in Step 1340 (e.g. 710) marked, and FIG. 11 shows an envelope for the chest respiratory signal after application of a smooth FIR filter in Step 1350 with starting points of respiratory phases determined in Step 1340 (e.g. 710) marked. Finally, FIG. 12 shows median amplitudes of the chest respiratory signal calculated in Step 1355 across numerous respiratory periods for two phase groups. As shown, the first phase group median amplitudes 1210 average 13.43, whereas the second phase group median amplitudes 1220 average 6.26, which indicates that the first phase group consists of inspiratory phases and the second phase group consists of expiratory phases. It bears noting that the median amplitudes plotted in FIG. 12 are larger than those plotted in FIGS. 9-11 due to normalization of amplitudes plotted in FIGS. 9-11.

Next, average inspiratory time and fractional inspiratory time are determined using the respiratory phase classifications made in Step 1355 using the chest respiratory signal and the starting points determined in Step 1340 using the tracheal respiratory signal (1360). Returning momentarily to FIG. 7, the respiratory phase classifications made in Step 1355 are used to identify the starting points and end points of inspiratory phases. Times for individual inspiratory phases are then determined from which a median inspiratory time is determined. Similarly, the respiratory phase classifications made in Step 1355 are used to identify the starting points and end points of respiratory periods. Times for individual respiratory periods are then determined from which a median respiratory period is determined. Finally, fractional inspiratory time can be calculated as the median inspiratory time divided by the median respiratory period.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A time domain signal processing method, comprising the steps of:
determining starting points for a plurality of respiratory phases using a tracheal respiratory signal; and
classifying the respiratory phases into inspiratory and expiratory phases using the starting points and a chest respiratory signal.

2. The method of claim 1, further comprising the step of determining respiratory time data using the starting points and the classified respiratory phases.

3. The method of claim 2, wherein the respiratory time data comprise one or more of average inspiratory time or average fractional inspiratory time.

4. The method of claim 1, further comprising the step of applying a band-pass filter to the tracheal respiratory signal.

5. The method of claim 1, further comprising the step of applying a smooth finite impulse response (FIR) filter to the tracheal respiratory signal.

6. The method of claim 1, further comprising the step of down-sampling the tracheal respiratory signal.

7. The method of claim 1, further comprising the step of applying an autocorrelation function to the tracheal respiratory signal.

8. The method of claim 1, further comprising the step of applying a band-pass filter to the chest respiratory signal.

9. The method of claim 1, further comprising the step of applying a smooth FIR filter to the chest respiratory signal.

10. The method of claim 1, further comprising the steps of collecting and amplifying the tracheal respiratory signal and the chest respiratory signal.

11. A time domain signal processing method, comprising the steps of:
determining starting points for a plurality of respiratory phases using a respiratory signal from a first body position;
classifying the respiratory phases into inspiratory and expiratory phases using the starting points and a respiratory signal from a second body position; and
determining respiratory time data using the starting points and the classified respiratory phases.

12. The method of claim 11, wherein the first body position is the trachea and the second body position is the chest.

13. The method of claim 11, wherein the first body position is the trachea and the second body position is the back.

14. The method of claim 11, wherein the respiratory time data comprise one or more of average inspiratory time or average fractional inspiratory time.

15. A respiratory signal detection and signal processing system, comprising:
first and second respiratory sound transducers adapted to collect first and second collected respiratory signals from first and second body positions, respectively; and
a time domain signal processor adopted to receive first and second received respiratory signals based on the first and second collected respiratory signals, respectively, wherein the time domain signal processor determines starting points for a plurality of respiratory phases using the first received respiratory signal, classifies the respiratory phases into inspiratory and expiratory phases using the starting points and the second received respiratory signal and determines respiratory time data using the starting points and the classified respiratory phases.

16. The system of claim 15, wherein the first collected respiratory signal comprises a tracheal respiratory signal and the second collected respiratory signal comprises a chest respiratory signal.

17. The system of claim 15, wherein the respiratory time data comprise one or more of average inspiratory time or average fractional inspiratory time.

18. The system of claim 15, wherein the time domain signal processor is collocated with at least one of the first or second sound transducer.

19. The system of claim 15, wherein the time domain signal processor is located remotely from the first and second sound transducers.
